(19) United States
(12) Patent Application Publication
(10) Pub. No.: US 2015/0025085 A1
(43) Pub. Date: Jan. 22, 2015

(54) Iron Chelators And Uses Thereof

(71) Applicant: Varinel Inc., West Chester, PA (US)

(72) Inventors: Vincent R. Zurawski, JR., Westtown, PA (US); Theodore J. Nitz, Boyds, MD (US)

(21) Appl. No.: 14/375,776

(22) PCT Filed: Jan. 30, 2013
(86) PCT No.: PCT/IB13/50789

§ 371 Date: Jul. 30, 2014

(30) Foreign Application Priority Data
Jan. 31, 2012 (US) PCT/US2012/023330

(51) Int. Cl. C07D 215/26

(52) U.S. Cl. CPC C07D 215/26
USPC 514/253.06; 544/363

(57) ABSTRACT

8-Hydroxy-quinoline derivatives and 8-ethers, 8-esters, 8-carbonates, 8-acyloxymethyl, 8-phosphates, 8(phosphoryloxy)methyl, and 8-carbamates derivatives thereof substituted at the 5-position are described as useful for iron chelation and neuroprotective therapies.

### FIELD OF THE INVENTION

The present invention relates to novel iron chelators, in particular to 8-hydroxy-5-substituted-quinolines and their pharmaceutical uses.

### BACKGROUND OF THE INVENTION

Iron is known to enhance the production of the highly reactive and toxic hydroxyl radical, thus stimulating oxidative damage. Studies in relevant animal models have shown a linkage between hydroxyl and oxygen free radicals production and neurodegenerative diseases and disorders, such as Parkinson's diseases, Alzheimer's disease, stroke, amyotrophic lateral sclerosis (ALS), multiple sclerosis, Friedreich's ataxia, neurodegeneration with brain iron accumulation (NBIA) disease, epilepsy, neurotrauma, and age-related macular degeneration (AMD).

Iron is also an essential co-factor for living organisms, particularly for bacteria which cannot grow unless they have a source of iron in the environment from which they can obtain the iron they need. Some pathogenic bacteria secrete small molecules called siderophores, which bind to and secrete iron from the environment and bring it back inside the bacteria where it is critical in chemical reactions for the continuing function and growth of the bacteria. Therefore, if the iron can be taken out with the aid of a chelator, the bacteria will become stressed and will be more susceptible to antibiotics.

One of the main problems in the use of chelating agents as antioxidant-type drugs for treatment of neurodegenerative diseases or as antibacterial is the limited transport of these ligands or their metal complexes through cell membranes or other biological barriers. Drugs with the brain as the site of action should, in general, be able to cross the blood brain barrier (BBB) in order to attain maximal in vivo biological activity.

8-Hydroxyquinoline is a strong chelating agent for iron and contains two aromatic rings, which can scavenge free radicals by themselves. PCT Publication WO 00/74664 and U.S. Pat. No. 6,855,711 disclose 8-hydroxyquinoline compounds as being useful for treatment of neurodegenerative disorders including Parkinson's disease and stroke. The lead compound, 5-[4-(2-hydroxyethyl)piperazin-1-ylmethyl]-8-hydroxyquinoline, designated VK28, was able to cross the BBB and was shown to be active against 6-qhydroxydopamine (6-OHDA) in an animal model of Parkinson's disease. PCT Publication WO 2004/041151 and U.S. Pat. No. 8,058,442 disclose 8-hydroxyquinoline iron chelator comprising a residue selected from a residue that imparts a neuroprotective function to the compound, a residue that imparts combined antiapoptotic and neuroprotective function to the compound, or both. The lead compound, designated M30, was also able to cross the BBB and to be active against 6-OHDA in an animal model of Parkinson's disease. WO 2010/086860 discloses multifunctional 8-hydroxyquinoline iron chelators designed to be able to cross the BBB.

It would be very desirable to provide novel iron chelators that exhibit also neuroprotective activity, good transport properties through cell membranes including the blood brain barrier, optimal oral uptake and optimal or sufficient oral uptake and PK behavior that would qualify them as drug candidates for clinical development.

### SUMMARY OF THE INVENTION

The present invention relates to 8-hydroxy-5-substituted-quinoline derivatives of Formula I described hereinafter and pharmaceutically acceptable salts thereof. In certain embodiments, the compounds of the invention are those of Formula II herein.

The compounds of Formula I are multifunctional compounds useful as iron chelators, as neuroprotective in the treatment of neurodegenerative diseases and disorders and as antibacterial.

In another aspect, the present invention provides a pharmaceutical composition comprising a compound of the invention or a pharmaceutically acceptable salt thereof and a pharmaceutically acceptable carrier.

In a further aspect, the present invention relates to a method for preventing and/or treating conditions, disorders or diseases that can be prevented and/or treated by iron chelation therapy and/or neuroprotective therapy, said method comprises administering to an individual in need thereof an effective amount of a compound of the invention or a pharmaceutical composition comprising same.

### BRIEF DESCRIPTION OF THE FIGURES

FIG. 1 shows inhibition of growth of Acinetobacter baumannii, strain 5711 by compound 4 herein at various concentrations.

### DETAILED DESCRIPTION OF THE INVENTION

In one aspect, the present invention relates to a compound of the formula I:

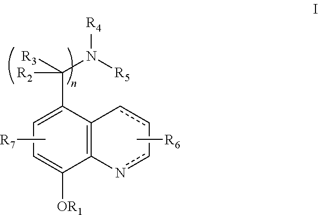

wherein

R₁ is selected from:
(i) H;
(ii) C₁-C₈ alkyl substituted by one or more radicals selected from hydroxy, C₁-C₈ alkoxy, cyano, carboxy, aminocarbonyl, C₁-C₈ alkylaminocarbonyl, di(C₁-C₈)alkylaminocarbonyl, and C₁-C₈alkoxycarbonyl;
(iii) —COR_{S}, wherein R₈ is C₁-C₈ alkyl, C₂-C₈ alkenyl, C₂-C₈ alkynyl, C₃-C₈ cycloalkyl, aryl, heteroaryl, or heterocyclyl wherein said alkyl, alkenyl, alkynyl, aryl, heteroaryl, or heterocyclyl group is optionally substituted by one or more of the following groups: halogen atoms, C₁-C₈ alkyl, hydroxy, amino, C₁-C₈ alkylamino, di(C₁-C₈)alkylamino, mercapto, C₁-C₈ alkylthio, cyano, C₁-C₈ alkoxy, carboxy, C₁-C₈ (alkoxy)carbonyl, C₁-C₈ (alkyl)carbonyloxy, C₁-C₈ (alkyl)sulfonyl, C₁-C₈ (alkyl)carbonylamino, aminocarbonyl, C₁-C₈ (alkyl)aminocarbonyl, or di(C₁-C₈)alkylaminocarbonyl, or a straight or branched C₁-C₅ alkyl may be substituted by amino at the α-position to the CO group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue.
(iv) —COOR_{S}, wherein R₉ is C₁-C₈ alkyl optionally substituted by halogen, C₁-C₈ alkoxy, phenyl optionally substituted by nitro, hydroxy, carboxy, or C₃-C₆ cycloalkyl; C₂-C₄ alkenyl; C₂-C₄ alkynyl; C₅-C₇ cycloalkyl; or phenyl optionally substituted by halogen, amino, nitro, C₁-C₈ alkyl, C₁-C₈ (alkoxy)carbonyl, or C₁-C₈ alkoxy;
(v) —CH₂—O—CO—R₁₀, or —CH(CH₃)—O—CO—R₁₀, wherein R₁₀ is C₁-C₈ alkyl optionally substituted by halogen, C₁-C₈ alkoxy; C₂-C₄ alkenyl optionally substituted by phenyl; C₃-C₆ cycloalkyl; phenyl optionally substituted by C₁-C₈ alkoxy; or heteroaryl selected from furyl, thienyl, isoxazolyl, or pyridyl optionally substituted by halogen or C₁-C₈ alkyl;
(vi) —PO(OR₁₁)₂, —CH₂—O—PO(OR₁₁)₂ or —CH(CH₃)—O—PO(OR₁₁)₂, wherein R₁₁ is independently selected from H, C₁-C₈ alkyl, or C₁-C₈ alkyl optionally substituted by hydroxy, C₁-C₈ alkoxy, or C₁-C₈ (alkyl)carbonyloxy; and
(vii) —CONR₁₂R₁₃, wherein R₁₂ and R₁₃ are independently selected from H, C₁-C₈ alkyl, C₂-C₈ alkenyl, C₂-C₈ alkynyl, C₃-C₈ cycloalkyl, aryl, arylalkyl, heteroaryl, or heterocyclyl wherein said alkyl, alkenyl, alkynyl, aryl, heteroaryl, heteroarylalkyl, heterocyclyl or heterocyclylalkyl group is optionally substituted by one or more of the groups: halogen atoms, C₁-C₈ alkyl, hydroxy, amino, C₁-C₈ alkylamino, di(C₁-C₈)alkylamino, mercapto, C₁-C₈ alkylthio, cyano, C₁-C₈ alkoxy, carboxy, C₁-C₈ (alkoxy)carbonyl, C₁-C₈ (alkyl)carbonyloxy, C₁-C₈ (alkyl)sulfonyl, C₁-C₈ (alkyl)carbonylamino, aminocarbonyl, C₁-C₈ (alkyl)aminocarbonyl, and di(C₁-C₈)alkylaminocarbonyl, or a straight or branched C₁-C₅ alkyl may be substituted by a carboxy group at the α-position to the amino group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue. or R₁₂ and R₁₃ together with the N atom to which they are attached form a 5 to 7 membered saturated ring optionally further containing a heteroatom selected from O, S and N, optionally substituted by C₁-C₈ alkyl;
R₂ and R₃ each independently is selected from a group consisting of H, C₁-C₈ alkyl, halogen, halo(C₁-C₈)alkyl, OH, C₁-C₈ alkoxy, amino, C₁-C₈ alkylamino, di(C₁-C₈)alkylamino, C₁-C₈ (alkyl)carbonylamino, carboxy, or C₁-C₈ (alkyl)carbonyloxy;
R₄ and R₅ together with the nitrogen atom to which they are attached form a 5-8 membered heterocyclic ring that may contain one or more nitrogen, oxygen, or sulfur atoms and may be optionally substituted at any available position in the ring with one or more radicals selected from the group consisting of H, C₁-C₈ alkyl, halogen, halo(C₁-C₈)alkyl, cyano, cyano(C₁-C₈)alkyl, (C₁-C₈)alkoxy, (C₁-C₈)alkoxy(C₁-C₈)alkyl, hydroxy, hydroxy(C₁-C₈)alkyl, amino, (C₁-C₈)alkylamino, di(C₁-C₈)alkylamino, amino(C₁-C₈)alkyl, (C₁-C₈)alkylamino(C₁-C₈)alkyl, di(C₁-C₈)alkylamino(C₁-C₈)alkyl, oxo, formyl, acyl, carboxy, (C₁-C₈)alkoxycarbonyl, carboxy(C₁-C₈)alkyl, acyloxy, acyloxy(C₁-C₈)alkyl, acylamino, acylamino(C₁-C₈)alkyl, (C₁-C₈)alkylsulfonyl, and arylsulfonyl radicals;
R₆ is H, C₁-C₈ alkyl, mercapto, C₁-C₈ alkylthio, amino, C₁-C₈ alkylamino, C₁-C₈ alkylimino, di(C₁-C₈)alkylamino, hydroxy, or C₁-C₈ alkoxy; or imino, oxo or thioxo at the 2- or 4-positions;
R₇ is H, halogen, C₁-C₈ alkyl, C₃-C₈ cycloalkyl, halo(C₁-C₈)alkyl, cyano, (C₁-C₈)alkoxy, hydroxy, amino, (C₁-C₈)alkylamino, di(C₁-C₈)alkylamino, nitro, acyloxy, acylamino, (C₁-C₈)alkylthio, (C₁-C₈)alkylsulfenyl, or (C₁-C₈)alkylsulfonyl;
each of the dotted lines indicates an optional bond; and
n is an integer from 1 to 8,
   and pharmaceutically acceptable salts thereof,
but excluding the compound wherein R₁, R₂, R₃, R₆, R₇ are H; n is 1; and R₄ and R₅ together with the N atom to which they are attached form a piperazino ring substituted at the 4-position by 2-hydroxyethyl.

In certain embodiments, the present invention relates to the compounds of the formula I wherein R₁ is H.

In certain embodiments, R₄ and R₅ together with the N atom to which they are attached form a piperazino ring that may substituted at the 4 position and the compound has the formula II below:

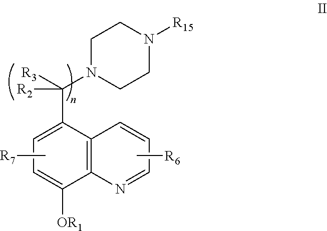

wherein

R₁, R₂, R₃ and R₇ each is as defined in claim 1;

R₆ is H, C₁-C₈ alkyl, mercapto, C₁-C₈ alkylthio, amino, C₁-C₈ alkylamino, C₁-C₈ alkylimino, di(C₁-C₈)alkylamino, hydroxy, or C₁-C₈ alkoxy;

R₁₅ is H, C₁-C₈ alkyl, halogen, halo(C₁-C₈)alkyl, cyano, cyano(C₁-C₈)alkyl, (C₁-C₈)alkoxy, (C₁-C₈)alkoxy(C₁-C₈)alkyl, hydroxy, hydroxy(C₁-C₈)alkyl, amino, (C₁-C₈)alkylamino, di(C₁-C₈)alkylamino, amino(C₁-C₈)alkyl, (C₁-C₈)alkylamino(C₁-C₈)alkyl, di(C₁-C₈)alkylamino(C₁-C₈)alkyl, oxo, formyl, acyl, carboxy, carboxy(C₁-C₈)alkyl, (C₁-C₈)alkyloxycarbonyl, acyloxy, acyloxy(C₁-C₈)alkyl, acylamino, acylamino(C₁-C₈)alkyl, (C₁-C₈)alkylsulfonyl or arylsulfonyl,

n is an integer from 1 to 8, and

pharmaceutically acceptable salts thereof, but excluding the compound wherein R₁, R₂, R₃, R₆, R₇ are H; n is 1 and R₁₅ is 2-hydroxyethyl.

In certain embodiments, the compounds of the invention are the compounds of formula II wherein R₁₅ is 2-hydroxyethyl and in particular the compounds wherein R₁ is H, R₂, R₃, R₆ and R₇ each is as defined above, R₁₅ is 2-hydroxyethyl and n is an integer from 2 to 5, preferably 2 or 3.

In certain embodiments, the compound of the invention has the formula II wherein R₁, R₂, R₃, R₆ and R₇ each is H, R₁₅ is hydroxyethyl, and n is 2, herein identified as compound 4. In other certain embodiments, the compound of the invention has the formula II wherein R₁, R₂, R₃, R₆ and R₇ each is H, R₁₅ is hydroxyethyl, and n is 3, herein identified as compound 6. In further certain embodiments, the compound of the invention has the formula II wherein R₁, R₂, R₃, and R₆ each is H, R₇ is F at the 7-position, R₁₅ is hydroxyethyl, and n is 2, herein identified as compound 5.

The term “halogen” as used herein refers to fluoro, chloro, bromo and iodo, and is preferably Cl or F.

The term “C₁-C₈ alkyl”, alone or as part of a radical containing an alkyl group, typically means a straight or branched alkyl having 1 to 8, preferably 1 to 6, 5, 4, 3, 2 or 1 carbon atoms and includes, without being limited to, methyl, ethyl, n-propyl, isopropyl, n-butyl, sec-butyl, isobutyl, tert-butyl, n-pentyl, 1-methylbutyl, 2,2-dimethylpropyl, n-hexyl, n-heptyl, n-octyl, and the like. The alkyl radical may be substituted, without being limited to, by one or more OH, SH, COOH, CONH₂, CN, cycloalkyl (e.g., cyclohexyl, optionally substituted by alkyl), aryl (e.g., phenyl, optionally substituted by NO₂), alkoxy, alkoxycarbonyl, alkylcarbonyloxy, and heteroaryl or heterocyclyl (e.g., furyl, thienyl, piperidino). The term “halo(C₁-C₈)alkyl” refers to C₁-C₈ alkyl, preferably C₁-C₅ alkyl substituted by one or more F atoms or by one or more F and Cl atoms. In certain embodiments the haloalkyl is pentafluoropentyl. In certain embodiments, the haloalkyl is methyl substituted by 1, 2 or 3 F atoms or by F and Cl such as —CH₂F, —CHF₂, —CF₃, or —CClF₂. In certain embodiments, the haloalkyl is ethyl substituted by 1 to 5 F atoms such as —CHFCH₃, —CF₂CH₃, —CF₂CFH₂, —CF₂CF₂H, —CH₂CF₃, or —CF₂CF₃.

The terms “C₂-C₈ alkenyl” and “C₂-C₈ alkynyl” typically mean a straight or branched radical having 2-8, preferably 2, 3 or 4, carbon atoms and one double or triple bond, respectively, and include, without being limited to, vinyl, allyl, prop-1-en-1-yl, prop-2-en-1-yl, but-3-en-1-yl, 2,2-dimethylvinyl, 2-ethenylbutyl, oct-3-en-1-yl, and the like, and ethynyl, propargyl, but-3-yn-1-yl, pent-3-yn-1-yl, and the like. The alkenyl radical may be substituted, for example, by aryl, e.g., phenyl.

The terms “C₁-C₈ alkoxy” and “C₁-C₈ alkylthio” as used herein typically mean a straight or branched radical having 1-8, preferably 1, 2, or 3 carbon atoms, and being preferably a substituent of an alkyl, phenyl or heteroaryl radical. Examples of alkoxy include methoxy, ethoxy, propoxy, isopropoxy, butoxy, pentoxy, and the like and of alkylthio include methylthio, ethylthio, propylthio, isopropylthio, butylthio and the like.

The term “acyl” alone or as part of a radical containing an acyl group refers to a C₂-C₁₉ (alkyl)carbonyl, C₂-C₁₉ (alkenyl)carbonyl, C₂-C₁₉ (alkynyl)carbonyl, C₃-C₈ (cycloalkyl)carbonyl, C₆-C₁₄ (aryl)carbonyl, heteroarylcarbonyl, or heterocyclylcarbonyl radical. Examples of such radicals include, without limitation, acetyl, benzoyl, caproyl, myristoyl, stearoyl, oleoyl, and the like. All the radicals of the acyl groups may be substituted as defined herein for alkyl, alkenyl, alkynyl, cycloalkyl, aryl, heteroaryl, or heterocyclyl.

The term “C₃-C₈ cycloalkyl” refers herein to a cycloalkyl radical comprising one or more rings such as cyclopropyl, cyclobutyl, cyclopentyl, cyclohexyl, cycloheptyl, cyclooctyl, adamantyl, bicyclo[3.2.1]octyl, bicyclo[2.2.1]heptyl, and the like, that may be substituted, for example, by one or more alkyl groups.

The term “aryl” refers to a C₆-C₁₄ aryl, namely, to an aromatic carbocyclic group having 6 to 14 carbon atoms consisting of a single ring or multiple rings either condensed or linked by a covalent bond such as, but not limited to, phenyl, naphthyl, carbazolyl, phenanthryl, and biphenyl. In certain embodiments, the aryl radical is phenyl optionally substituted by halogen, C₁-C₈ alkyl, C₁-C₈ alkoxy, nitro, C₃-C₈ cycloalkyl, cyano, hydroxy, mercapto, (C₁-C₈)alkylthio, (C₁-C₈)alkylsulfenyl, (C₁-C₈)alkylsulfonyl, carboxy, (C₁-C₈)alkoxycarbonyl, (C₁-C₈)alkylcarbonyl, amino, (C₁-C₈)alkylamino, di(C₁-C₈)alkylamino, formyl, aminocarbonyl, (C₁-C₈)alkylaminocarbonyl, di(C₁-C₈)alkylaminocarbonyl, acylamino, and/or (C₁-C₈)alkylsulfonylamino. In some preferred embodiments, the aryl radical is phenyl, optionally substituted by halogen, e.g., F, alkyl, e.g., methyl, alkoxy, e.g., methoxy, and/or nitro.

The term “heteroaryl” refers to a radical derived from a mono- or poly-cyclic heteroaromatic ring containing one to three heteroatoms selected from the group consisting of N, O and S. When the heteroaryl is a monocyclic ring, it is preferably a radical of a 5-6-membered ring such as, but not limited to, pyrrolyl, furyl, thienyl, pyrazolyl, imidazolyl, oxazolyl, isoxazolyl, thiazolyl, isothiazolyl, pyridyl, pyrimidinyl, 1,3,4-triazinyl, 1,2,3-triazinyl, and 1,3,4-triazinyl. Polycyclic heteroaryl radicals are preferably composed of two rings such as, but not limited to, benzofuryl, isobenzofuryl, benzothienyl, indolyl, quinolinyl, isoquinolinyl, imidazo[1,2-a]pyridyl, benzimidazolyl, benzothiazolyl and benzoxazolyl. It is to be understood that when a polycyclic heteroaromatic ring is substituted, the substitutions may be in any of the carbocyclic and/or heterocyclic rings. In some embodiments, the heteroaryl is furyl, thienyl, isoxazolyl, pyridyl (optionally substituted by Cl), indolyl, or imidazolyl.

The term “heterocyclyl” refers to a radical derived from a mono- or poly-cyclic non-aromatic ring containing one to three heteroatoms selected from the group consisting of N, O and S. Examples of such radicals include, without limitation, piperidinyl, 4-morpholinyl, pyrrolidinyl.

As used herein, “n” is an integer from 1 to 8, preferably from 1 to 5. In certain embodiments, n is 1, 2 or 3.

The compounds of the invention of the formula II are derivatives or analogs of 5-((4-(2-hydroxyethyl)piperazin-1-yl)methyl)-8-hydroxyquinoline (also identified herein as VK28), which was shown in WO 00/74664 and U.S. Pat. No. 6,855,711 to be able to cross the BBB and to be active against 6-hydroxydopamine (6-OHDA) in an animal model of Parkinson's disease thus indicating its potential usefulness for treatment of neurodegenerative disorders including Parkinson's disease and stroke. VK28 and its analog compound 4 were found to have antibacterial activity and methods and compositions comprising VK28 or compound for treating bacterial infections are described in PCT Application No. PCT/US2012/023377 (WO 2012/106364) entitled “Methods and compositions for treating bacterial infections with iron chelators”, filed on Jan. 31, 2012 and assigned to the Government of the United States, as represented by the Secretary of the Army.

Derivatives of VK28 according to the present invention include compounds of formula II wherein R₂, R₃, R₆ and R₇ each is H, R₁₅ is 2-hydroxyethyl, n is 1 and R₁ is not H, namely, the 8-hydroxy group of the quinoline ring is modified as defined above for the compounds of formula I. Preferably, the 8-OH is modified in such a way that it still maintains its iron chelating function. Other derivatives include the compounds of formula II wherein R₆ and/or R₇ are different from H or R₁₅ is different from 2-hydroxyethyl.

Analogs of VK28 according to the present invention include compounds of formula II wherein n is an integer from 2 to 8, preferably 2 to 5, more preferably 2 or 3, namely the piperazine ring is linked to the 5-position of the quinoline ring by a —(CH₂)ₙ chain, wherein n is 2 to 8.

In certain embodiments, the derivatives are 8-ethers of VK28, wherein R₁ is C₁-C₈ alkyl as defined above in (ii) and is preferably C₁-C₃ alkyl substituted by hydroxy or C₁-C₃ alkoxy, more preferably R₁ is hydroxypropyl, methoxypropyl or propoxymethyl.

In certain embodiments, when R₁ is —COR₈ and R₈ is a C₁-C₅ alkyl group substituted by an amino group at the α-position to the CO group and optionally further substituted by a group selected from hydroxy, methylthio, mercapto, phenyl, 4-hydroxyphenyl, indolyl, aminocarbonyl, carboxy, amino, guanidino, and imidazolyl. The residue of proline is formed when R₈ is defined as heterocyclyl consisting of 2-pyrrolidinyl.

In certain embodiments, the derivatives are 8-esters of VK-28, wherein R₁ is —COR₈ as defined above in (iii). In certain embodiments, R₈ is C₁-C₅ alkyl, for example, methyl optionally substituted by methoxy, methoxycarbonyl, carboxy, methylcarbonyloxy or one or more of Cl or F atoms, e.g. methoxymethyl, methoxycarbonylmethyl, methylcarbonyloxymethyl, chloromethyl or trifluoromethyl, or ethyl optionally substituted by ethoxy, isobutyl, or sec-pentyl. In certain embodiments, R₈ is C₂-C₄ alkenyl, preferably vinyl optionally substituted by phenyl (e.g. 2-phenylvinyl), 1-methylvinyl, 2-methylvinyl, 2,2-dimethylvinyl, or but-3-en-1-yl. In certain embodiments, R₈ is C₃-C₅ cycloalkyl such as cyclopropyl or cyclopentyl; aryl such as phenyl optionally substituted by methoxy; preferably at position 4; heteroaryl such as 2-thienyl, 2-furyl, 5-isoxazolyl or pyridyl optionally substituted by Cl; or heterocyclyl such as 4-morpholinyl. In certain embodiments, R₈ is a straight or branched C₁-C₅ alkyl substituted by amino at the α-position to the CO group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue.

In certain embodiments, the derivatives are 8-carbonates of VK-28, wherein R₁ is —COOR₉ as defined above in (iv). In certain embodiments, R₉ is C₁-C₈ alkyl such as methyl optionally substituted by Cl, 4-nitrophenyl or C₆ cycloalkyl, ethyl optionally substituted by methoxy, e.g. 2-methoxyethyl, or one or more Cl or F atoms, e.g. 1-chloroethyl, 2-chloroethyl, 2-fluoroethyl, 2,2,2-trichloroethyl, or 2,2,2-trifluoroethyl, propyl, butyl, isobutyl, pentyl, or octyl; C₂-C₃ alkenyl such as vinyl, 1-methylvinyl or allyl; C₃-C₄ alkynyl such as propargyl or but-3-yn-yl; C₅-C₆ cycloalkyl such as cyclopentyl or cyclohexyl; or phenyl optionally substituted by nitro, fluoro, methoxy or methyl such as 4-nitrophenyl, 4-fluorophenyl, 4-methoxyphenyl, or 4-methylphenyl.

In certain embodiments, the derivatives are 8-acyloxymethyl derivatives of VK-28, wherein R₁ is —CH₂—O—CO—R₁₀, or —CH(CH₃)—O—CO—R₁₀ as defined above in (v). In certain embodiments, R₁₀ is C₁-C₅ alkyl such as methyl optionally substituted by methoxy, methoxycarbonyl, methylcarbonyloxy, or one or more Cl or F atoms, e.g. chloromethyl of trifluoromethyl, ethyl optionally substituted by ethoxy, isobutyl, or 1-methylbutyl; C₂-C₄ alkenyl such as vinyl optionally substituted by phenyl, e.g. 2-phenylvinyl, 1-methylvinyl, 2-methylvinyl, 3-buten-1-yl, or 2,2-dimethylvinyl; C₃-C₅ cycloalkyl such as cyclopropyl or cyclopentyl; phenyl optionally substituted by methoxy such as 4-methoxyphenyl; or heteroaryl such as 2-furyl, 2-thienyl, 5-isoxazolyl, or pyridyl optionally substituted by halogen such as 2-chloro-pyrid-5-yl.

In certain embodiments, the derivatives are 8-phosphates or (phosphoryloxy)methyl derivatives of VK-28, wherein R₁ is —PO(OR₁₁)₂, —CH₂—O—PO(OR₁₁)₂ or —CH(CH₃)—O—PO(OR₁₁)₂, as defined in (vi) above.

In certain embodiments, the derivatives are 8-carbamate derivatives of VK-28, wherein R₁ is —CONR₁₂R₁₃ as defined in (vii) above. When R₁₂ or R₁₃ is a C₁-C₅ alkyl group substituted by a carboxy group at the α-position to the —CON— group and optionally further substituted by a group selected from hydroxy, methylthio, mercapto, phenyl, 4-hydroxyphenyl, indolyl, aminocarbonyl, carboxy, amino, guanidino, and imidazolyl, the radical formed is a residue of a natural amino acid that typically occurs in proteins, including glycine, alanine, valine, leucine, isoleucine, lysine, valine, phenylalanine, glutamic acid, aspartic acid, asparagine, glutamine, arginine, histidine, proline, serine, tyrosine, methionine, threonine, and tryptophan. The residue of proline is formed when one of R₁₂ or R₁₃ is defined as heterocyclyl consisting of 2-pyrrolidinyl.

In certain embodiments, the compound of the invention is a derivative or analog of VK-28 modified at the quinoline ring. In certain embodiments, one or both of the carbocyclic ring or the heterocyclic ring of the quinoline structure may be hydrogenated, as indicated by the dotted lines in Formula I above.

In certain embodiments, the quinoline ring may be substituted at either of the rings, for example, at the 6 or 7 position by a group R₇; or at any of the 2, 3 or 4 positions by R₆ which may be C₁-C₈ alkyl, C₁-C₈ alkoxy, C₁-C₈ alkylthio, hydroxy, mercapto, amino, C₁-C₈ alkylamino, or di(C₁-C₈)alkylamino, and when the heterocyclic ring is partially hydrogenated, R₆ may also be oxo, thioxo, imino, or C₁-C₈ alkylimino.

In certain embodiments, the derivatives or analogs of VK-28 are substituted at the methylene group (n=1) at the 5-position, namely, R₂ and/or R₃ may be C₁-C₈, preferably C₁-C₃ alkyl, more preferably methyl; halogen, preferably F; or —CF₃. Examples of such compounds include the compounds of formula II wherein R₁, R₂, R₆ and R₇ are H, n is 1, R₃ is CH₃ or CF₃, and R₁₅ is hydroxyethyl, and compound wherein R₁, R₆ and R₇ are H, and R₂ and R₃ each is F.

In certain embodiments, the analogs of VK-28 are compounds of formula I or II wherein the methylene radical at the 5-position is replaced by an ethylene or propylene radical, namely, n is 2 or 3. Examples of such compounds include the compounds of formula II, herein identified as compounds 4 and 5, wherein n is 2, R₁, R₂, R₃, and R₆ are H, and R₇ is H, or F, respectively, and R₁₅ is hydroxyethyl.

The present invention further relates to pharmaceutically acceptable salts of the compounds including salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, formate, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, ethanesulfonate, 2-hydroxyethanesulfonate, malate, salicylate, napsylate, hippurate, mucate, besylate, esylate and the like. Also contemplated are salts of amino acids such as arginate, aspartate and the like and gluconate or galacturonate (see, for example, Handbook of Pharmaceutical Salts, Properties, Selection, and Use, Stahl, P. H. and Wermuth, C. G., Eds; VHCA and Wiley-VCH: Zurich and Weinheim, 2002).

The compounds of Formula I herein, wherein n is 2-8, can be prepared by reacting 8-hydroxyquinoline with a ω-Cl acyl chloride of the formula: Cl—(CH₂)₁₋₇—COCl, thus obtaining a 5-(CO—(CH₂)₁₋₇—Cl)-8-hydroxyquinoline, reducing the CO group to CH₂, for example, with triethylsilane, and reacting the obtained 5-((CH₂)₁₋₈—Cl)-8-hydroxyquinoline with a cyclic amine represented by NR₄R₅. When n=2, the 8-hydroxyquinoline is reacted with chloroacetyl chloride, when n=3, with chloropropyonyl chloride, and so on. In certain embodiments, the amine NR₄R₅ is N-(2-hydroxyethyl)piperazine.

The compound wherein n=3 can be prepared by an alternative synthesis wherein 8-hydroxyquinoline is reacted with formaldehyde and concentrated HCl, reacting the obtained 5-(CH₂Cl)-8-hydroxyquinoline with diethylmalonate, hydrolyzing to the free dicarboxylic acid, heating for conversion to the 5-(CH₂)₂—COOH group, reacting with SOCl₂ and a reducing agent to obtain 5-((CH₂)₃—OH)— 8-hydroxyquinoline, which is converted to 5-((CH₂)₃Cl)-8-hydroxyquinoline and then reacted with a cyclic amine represented by NR₄R₅.

In certain embodiments, the compounds of Formula I are substituted at position 8 by a radical OR₁ wherein R₁ is different than H. This is a simple starting approach leading to the least alteration in structure, thereby preserving as many desirably properties of the parent compound having the phenolic 8-hydroxyl group. One strategy for assessing the suitability of a prodrug to enhance the bioavailability and PK of each of the compounds is preparation of simple esters (R₁ is COR₈) that gauge steric bulk as a factor in esterase hydrolysis. A resultant prodrug ester derivative is expected to provide for both better absorption and longer circulating t_{1/2}, depending upon the kinetics of plasma de-esterification known to be catalyzed by plasma esterases. In addition, esterification with amino acids may aid by amino acid active transport across the gut and the blood brain barrier. L-Valine is known to improve bioavailability of several drugs. Another strategy is etherification of the 8-hydroxyl group thus providing ether derivatives (R₁ is alkyl) prodrugs with a long-term stability in aqueous environment. A further strategy is to prepare carbonates (R₁ is COOR₉) that more readily expose the alcohol-leaving group to esterases and upon hydrolysis would spontaneously generate CO₂. These and other derivatives at the 8-position can be prepared by the methods described in WO 2012/020389 for similar compounds.

The compounds of the invention of Formulas I and II, particularly when R₁ is H, are specific iron chelators that are suitable to bind unbound iron within the cells. Iron that is not bound to transferrin is the toxic form of iron. The iron chelators of the invention have good transport properties and cross cell membranes thus chelating the unbound iron in excess within the cells. It is expected that their complexes with iron will leave the cells freely and will be rapidly excreted. It is further expected that the compounds, or at least a major part of the compounds, will be able to cross the BBB and thus will be suitable candidates for treatment of neurodegenerative diseases, disorders and conditions.

In another aspect, the present invention relates to a pharmaceutical composition comprising a compound of the formula I or II herein or a pharmaceutically acceptable salt thereof, and a pharmaceutically acceptable carrier, but excluding the compositions when used for antibacterial therapy and treatment of wounds caused by multidrug resistant bacteria.

The compounds, pharmaceutically acceptable salts and the pharmaceutical compositions of the invention are useful for preventing and/or treating conditions, disorders or diseases that can be prevented and/or treated by iron chelation therapy or by neuroprotective therapy.

In certain embodiments, the compounds are for use in the prevention and/or treatment of neurodegenerative and cerebrovascular diseases, conditions and disorders such as Parkinson's disease, Alzheimer's disease, stroke, amyotrophic lateral sclerosis (ALS), multiple sclerosis, Friedreich's ataxia, NBIA, epilepsy, and neurotrauma. The compounds can also be useful for promoting nerve regeneration, nerve restoration or to prevent or inhibit secondary degeneration which may otherwise follow primary nervous system injury, e.g., closed head injuries and blunt trauma, such as those caused by participation in dangerous sports, penetrating trauma, such as gunshot wounds, hemorrhagic stroke, ischemic stroke, glaucoma, cerebral ischemia, or damages caused by surgery such as tumor excision.

The iron chelator compounds I and II of the invention are useful for the treatment of Parkinson's disease and other metal-associated neurological disorders and for the treatment of trauma and stroke and the secondary injuries which follow them, by virtue of their ability to cross the blood brain barrier and to prevent lipid peroxidation in the brain, a process which leads to neuronal death.

The “prevention” aspect of the use of the iron chelators of the invention in diseases such as Parkinson's disease and Alzheimer's disease involves the prevention of further neurodegeneration and of the further progress of the disease.

In certain embodiments, the compounds of the invention can be used for prevention and/or treatment of the following diseases or disorders: age related macular degeneration; glaucoma; diabetes; iron overload in hemochromatosis and thalassemia; cardiovascular diseases, e.g. to prevent the damage associated with free radical generation in reperfusion injury; inflammatory disorders such as a joint inflammatory disorder, particularly rheumatoid arthritis, inflammatory bowel disease (IBD), and psoriasis; anthracycline cardiotoxicity, in case of cancer patients being treated with anthracycline neoplastic drugs; protozoal infection such as malaria caused by Plasmodium falciparum; yeast infection such as Candida albicans infection; viral infection such as retroviral infection, e.g., HIV-1, for the treatment of AIDS, optionally in combination with one or more antiviral agents such as abacavir, atazanavir, combivir, darunavir, fosamprenavir, indinavir, lopinavir, nelfinavir, raltegravir, ritonavir, saquinavir, tenofovir, tipranavir, trizivir, or zidovudine.

In certain embodiments, the compounds can be used for retarding ageing and/or improving the ageing process by prevention of ageing-related diseases, disorders or conditions such as neurodegenerative diseases, disorders or conditions; and for prevention and/or treatment of skin ageing and/or skin damage associated with ageing and/or exposure to sunlight and/or UV light.

In certain embodiments, the compounds can be used in cosmetic composition along with a cosmeticeutically acceptable carrier, useful for topical application for prevention and/or treatment of skin ageing and/or skin damage associated with ageing and/or exposure to sunlight and/or UV light. The cosmetic composition may be in the form of a lotion or cream and may be administered with other agents for skin treatment.

In certain embodiments, the iron chelators are for use ex-vivo for preservation of organs intended for transplantation such as heart, lung or kidney.

The present invention further relates to a method for iron chelation and neuroprotective therapy which comprises administering to an individual in need thereof an effective amount of a compound of the invention or of a pharmaceutically acceptable salt thereof.

In certain embodiments, the present invention provides a method for prevention and/or treatment of a viral, protozoal or yeast infection which comprises administering to an individual in need thereof an effective amount of a compound of the invention or of a pharmaceutically acceptable salt thereof, alone or in combination with an antiviral, antiprotozoan or antifungal drug. In certain embodiments, the viral infection is a retroviral infection, e.g. HIV-1, and the compound is used in the treatment of AIDS, optionally in combination with antiviral agents. In certain embodiments, the protozoal infection is malaria caused by Plasmodium falciparum. In certain embodiments, the yeast infection is a Candida albicans infection. For these uses, the compounds of the invention are those of Formula I or II herein wherein R₁ is H.

In certain embodiments, the present invention provides a method for retarding ageing and/or improving the ageing process by prevention of ageing-related diseases, disorders or conditions which comprises administering to an individual in need thereof an effective amount of a compound of the invention or of a pharmaceutically acceptable salt thereof. The individual in need may be a healthy individual or an individual suffering from an age-related disease such as a neurodegenerative disease, disorder or condition.

In certain embodiments, the present invention provides a method for prevention and/or treatment of skin ageing and/or skin damage associated with ageing and/or exposure to sunlight and/or UV light, which comprises administering to an individual in need thereof an effective amount of a compound of the invention or of a pharmaceutically acceptable salt thereof. The compound is most preferably administered topically in a pharmaceutical or cosmetic formulation.

For preparing the pharmaceutical compositions of the present invention, methods well-known in the art can be used. Inert pharmaceutically acceptable carriers can be used that are either solid of liquid. Solid form preparations include powders, tablets, dispersible granules, capsules, cachets and suppositories.

A solid carrier can be one or more substances which may also act as diluents, flavoring agents, solubilizers, lubricants, suspending agents, binders, or tablet disintegrating agents; it can also be an encapsulating material.

Liquid pharmaceutical compositions include solutions, suspensions, and emulsions. As an example, water or water-propylene glycol solutions for parenteral injection may be mentioned. Liquid preparations can also be formulated in solution in aqueous poly(ethylene glycol) solution. Aqueous solutions for oral use can be prepared by dissolving the active component or pharmaceutically acceptable salts thereof in water and adding suitable colorants, flavoring agents, stabilizers, and thickening agents as desired. Aqueous suspensions for oral use can be made by dispersing the finely divided active component in water with viscous material, i.e., natural or synthetic gums, resins, methyl cellulose, sodium carboxymethyl cellulose, and other well-known suspending agents. Formulations for topical application in the form of cream or gel are suitable for treatment of wounds caused by MDR bacteria.

Preferably, the pharmaceutical composition is in unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vial or ampoules. The unit dosage form can also be a capsule, cachet, or table itself or it can be the appropriate number of any of these packaged forms.

In therapeutic use for the treatment of Parkinson's disease, the compounds utilized in the pharmaceutical method of this invention may be administered to the patient at dosage levels of from 1 mg/kg to 20 mg/kg per day.

In therapeutic use for the treatment of stroke one or more dosages of from about 100 mg/kg to about 500 mg/kg of body weight may be administered to the patient as soon as possible after the event.

The dosage, however, may be varied depending upon the requirements of the patient, the severity of the condition being treated, and the compound being employed. Determination of optimum dosages for a particular situation is within the skill of the art.

The following examples illustrate particular methods for preparing compounds in accordance with this invention. These examples are intended as an illustration, and not as a limitation, of the scope of the invention.

### EXAMPLES

The following examples describe the structure of compounds of the invention (Chemical Section) and their biological activity (Biological Section). These examples are intended as an illustration, and not as a limitation, of the scope of the invention.

### I. Chemical Section

### Example 1

### Synthesis of 5-(2-(4-(2-hydroxyethyl)piperazin-1-yl)ethyl)-8-quinolinol dihydrochloride (herein designated as compound 4 dihydrochloride)

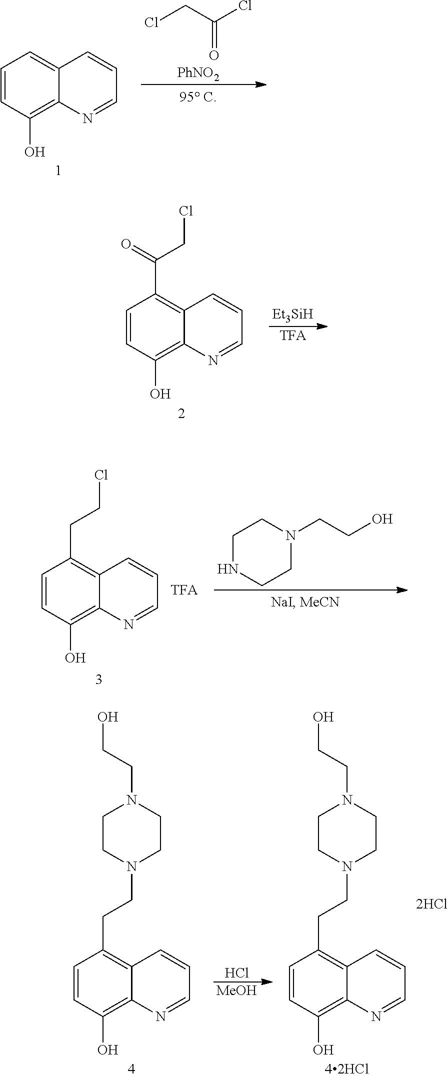

To a stirred solution of quinolin-8-ol (compound 1; 97 g, 0.669 mol, 1 eq.) in nitrobenzene (toxic and carcinogenic) was added chloro acetyl chloride (55.8 mL, 0.701 mol, 1.05 eq.) at 0° C. under argon, forming a yellow suspension. AlCl₃ (160 g, 1.2 mol, 1.79 eq.) was added portion wise and the suspension became clear upon stirring. The reaction mixture was heated to 100° C. for 36 hours. The reaction mixture was cooled down to room temperature and poured to mixture of HCl (450 mL, 6 N), ice (600 g) and methyl t-butyl ether (MTBE, 800 mL). The yellow precipitate was filtered via a filter paper, washed with MTBE (˜500 mL) and dried. The precipitate was digested to break up the aluminum complex with 200 mL of 12 N HCl at room temperature for 3 days, filtered and washed with ethyl acetate (EA). The resulting solid salt was stirred with 10% NaOAc aqueous solution (enough to make pH=6, ˜2 L used) to give a greenish suspension. The green precipitate was collected, dissolved in 1.8 L dichloromethane and dried over MgSO₄ anhydrous. Upon condensation, a yellow solid precipitated out, and the solution size was reduced to 500 mL. The yellow solid was filtered and washed with MTBE once and dried providing 58.5 g (38.5%) of compound 2 as a yellow solid.

Under argon protection, to a three neck round bottom flask containing compound 2 (58 g, 0.26 mol, 1 eq.) and chilled to 5° C. was added trifluoroacetic acid (TFA, 500 mL) followed by addition of triethylsilane (244 g, 2.1 mol, 8 eq.). The reaction was warmed to room temperature and heated to 60° C. overnight (16 hours). The mixture was cooled down to room temperature. The volatiles were first evaporated on rotary evaporator, and later using high vacuum at 30-40° C. The clear oil was decanted from the dark product residue and triturated with ether. The precipitated solid was filtered, rinsed with ether, and dried to provide 70 g (83.8%) of compound 3 as a yellow solid.

To a suspension of compound 3 (20 g, 62 mmol, 1 eq.) in anhydrous acetonitrile (200 mL) was added NaI (9.3 g, 62 mmol, 1 eq.) and 2-piperazin-1-yl-ethanol (140.4 g, 310 mmol, 5 eq.). The mixture was heated to 100° C. overnight (20 h) in a sealed reaction tube. The reaction mixture was cooled down to room temperature and the volatiles were evaporated. The residue was diluted with 500 mL water and extracted with dichloromethane (DCM) three times. The organic layer was washed with water, brine and dried over sodium sulfate anhydrous. The DCM solution was condensed to a crude brown solid. The resulting solid was dissolved in EA and triturated with hexanes to give 8.6 g (46%) of compound 4 as a yellow solid.

Compound 4 (8.35 g, 27.7 mmol, 1 eq.) was dissolved in methanol (200 mL) and HCl in ether (27.7 mL, 55.4 mmol, 2 eq.) was added. The mixture was stirred at room temperature overnight (16 h), and a yellow suspension was formed. To the reaction mixture was added ether (400 mL) under vigorous stirring. The yellow precipitate was collected and freeze dried to afford 10.3 g (99%) of the final compound 4.2HCl salt as a light yellow solid.

### Example 2

### Synthesis of 7-fluoro-5-(2-(4-(2-hydroxyethyl)piperazin-1-yl)ethyl)-8-quinolinol dihydrochloride (Herein Designated as Compound 5 dihydrochloride)

The synthesis is identical to that described in Example 1 for the synthesis of compound 4.2HCl employing 7-fluoro-8-quinolinol instead of 8-quinolinol as the starting material.

### Example 3

### Synthesis of 5-(3-(4-(2-hydroxyethyl)piperazin-1-yl)propyl)quinolin-8-ol dihydrochloride (Herein Designated as Compound 6 dihydrochloride)

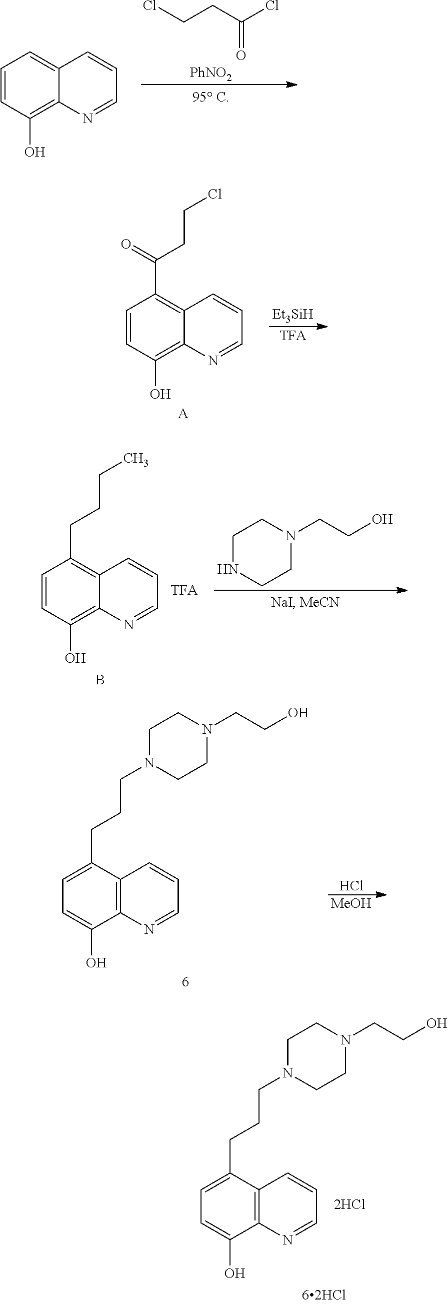

8-Quinolinol is reacted with 3-chloropropanoyl chloride to give 3-chloro-1-(8-hydroxyquinolin-5-yl)propan-1-one (compound A), which is reduced with triethylsilane in TFA to give 5-(3-chloropropyl)quinolin-8-ol (compound B). Compound B is then reacted with 2-piperazin-1-yl-ethanol under heating to give compound 6, which is dissolved in methanol and HCl in ether resulting in the final compound 6.2HCl salt.

### Example 4

### Alternative synthesis of 5-(3-(4-(2-hydroxyethyl)piperazin-1-yl)propyl)quinolin-8-ol dihydrochloride (Herein Designated as Compound 6 dihydrochloride)

Compound 6 can be synthesized using a different approach as shown below.

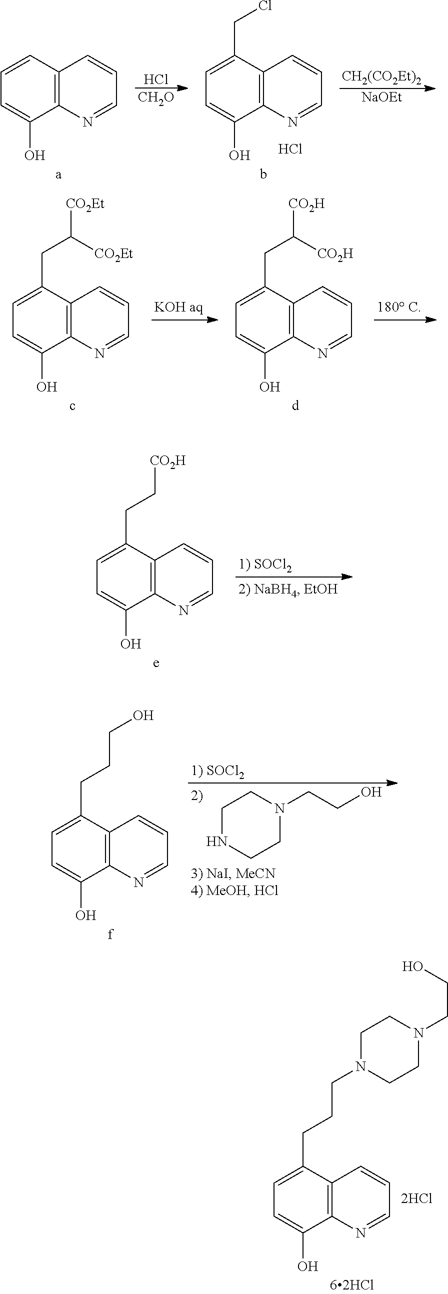

Hydrogen chloride gas was bubbled through a mixture of 8-quinolinol (compound a; 20 g, 138 mmol), aqueous formaldehyde (25 mL), and concentrated hydrochloric acid (37%, 25 mL) at 0° C. for 6 hours with stirring. The stirring was stopped and the reaction reached room temperature overnight. The resulting yellow solid was filtered and dried under vacuum at room temperature affording compound b.

Diethyl malonate (10 mL, 66 mmol, 5 eq) was added dropwise to a solution of sodium ethoxide (14.7 mL of 21 wt %, 39 mmol, 3 eq) in anhydrous EtOH (15 mL) at 0° C. with stirring. Compound b (3.0 g, 13 mmol) was added and the reaction mixture was allowed to warm to room temperature for 2 hours. The solvent was evaporated in vacuo and the resulting residue dissolved with water and ethyl acetate. The layers were separated and the aqueous layer extracted twice with ethyl acetate. The combined organic layers were washed twice with water, once with brine, and concentrated in vacuo. The material was purified by chromatography on silica gel with 10-30% ethyl acetate in hexanes. Product fractions were pooled and concentrated in vacuo and the resulting white solid dried under vacuum affording compound c.

A solution of potassium hydroxide (9.3 g) in water (15 mL) was added to compound c (3.0 g, 9.46 mmol) and the reaction mixture was stirred at room temperature over the weekend. The pH of the reaction was adjusted to between 4 and 5 with concentrated HCl and diluted with water (20 mL). The resulting yellow precipitate was filtered, rinsed with water, and dried under vacuum affording compound d.

Compound d was heated to 180° C., vented to a bubbler, for 1 hour and then allowed to cool to room temperature under argon, yielding compound e.

A suspension of compound e (1.0 g, 4.6 mmol) in thionyl chloride (9.0 mL) was stirred at room temperature for 0.5 h and then the volatiles were evaporated in vacuo. The resulting yellow solid was stirred with anhydrous DCM (5 mL) at 0° C. and then a solution of sodium borohydride (0.52 g, 14 mmol, 3 eq) in anhydrous EtOH (9 mL) was added. The reaction mixture was allowed to warm to room temperature for 1 hour then quenched with water. The aqueous mixture was extracted three times with 10% MeOH in DCM. The combined organic layers were washed with water, brine, and dried over anhydrous sodium sulfate. The material was purified by chromatography on silica gel with 20-60% ethyl acetate in hexanes. Product fractions were concentrated in vacuo and the resulting white solid dried under vacuum yielding compound f.

Compound f is reacted with thionyl chloride to obtain 5-(3-chloropropyl)quinolin-8-ol, which is then reacted with 2-piperazin-1-yl-ethanol using the method described for compound 4 in Example 1 to give compound 6, which is dissolved in methanol and HCl in ether resulting in the final compound 6.2HCl salt.

### II. Biological Section

### Methods

### (a) Metal Binding Properties

It is known that 8-quinolinol is a strong chelator for iron and has a higher selectivity for iron over copper. It is an important precondition for the antioxidative-type drugs because it is the excessive iron stores and iron-mediated generation of free radicals in the brain that are thought to be associated with neurodegenerative diseases. Therefore, only chelators with a higher selectivity for iron over copper are expected to chelate iron instead of copper and have potential neuroprotective effects. In order to discuss possible correlation between chelating properties of 8-quinolinol and its derivatives with their antioxidative ability, and the correlation between its derivative and the best established iron chelating drug, desferal, with antioxidative properties, a reliable measurement of the stability constants of the newly synthesized compounds is necessary. A spectrophotometric method is used for measurement of the iron-complexes stability constants of the compounds.

### (b) Mitochondria Isolation

Male Sprague-Dawley rats (300-350 g) are decapitated and the brains are immediately isolated and cooled in ice-cold isotonic 10 mM Tris-HCl buffer (pH 7.5) containing 0.25 M sucrose, 2 mM EDTA and 2% bovine serum albumin free of fatty acids (isolation buffer), and homogenized with 50 mL glass-teflon homogenizer with a motor (Heidolf, Germany) at 200 rpm in a 1:10 (w/v) ratio isolation buffer. The homogenate is centrifuged at 1000 g for 10 min and the resultant supernatant then centrifuged at 10,000 g for 10 min. The pellet is washed with 10 mM Tris-HCl (pH 7.5), 0.25 M sucrose, and centrifuged again at 10,000 g for 10 min. This step is then repeated three more times. The pellet is resuspended in 10 mM Tris-HCl (pH 7.5), 0.25 M sucrose at a final concentration of 50-60 mg protein/mL. The samples are stored at −18° C. until use.

### (c) Prevention of Lipid Peroxidation in Brain Tissue

Brain cortex homogenates (10% wt/vol) from male Wistar rats are prepared in 0.3 M sucrose and incubated in air. Aliquots (0.1 mL) of homogenate are incubated alone at 30° C. for 90 min to determine basal lipid peroxidation, or incubated after the addition of 10⁴ Fe₂(SO₄)₃ or FeCl₃ and in the presence of 10⁻³ M iron chelator of formula I or II. For the assay, to 0.3 mL of the homogenate there are added 0.2 mL of 8% SDS, 1.5 mL of 20% acetic acid pH 3.0-3.5, 1.5 mL of 0.8% thiobarbituric acid (TBA) and 0.5 mL of H₂O₂×2, the mixture is incubated at 95° C. for 60 min, cooled and lipid peroxidation is assayed by measurement of malondialdehyde formation at 532 nm. Standard curve: 1,1,3,3-tetraethoxypropane 0.1-25 nmol in 0.3 mL.

In another experiment, the ability of compounds 4, 5 and 6 to inhibit lipid peroxidation in vitro as initiated by iron and ascorbate is examined in brain mitochondria preparation employing the malonaldehyde procedure.

The experiments are carried out in triplicates. 7.5 μM of mitochondrial preparation (0.25 mg protein) are suspended in 750 μM of 25 mM Tris-HCl (pH 7.4) containing 25 pM ascorbic acid. Samples of the drugs to be tested are dissolved in water or ethanol and added to the suspension. The reaction is started by the addition of 2.5 or 5 μM FeSO₄ (from a 1 mM stock solution), and incubation for 2 h at room temperature. The reaction is stopped by addition of 750 μL of 20% (w/v) trichloroacetic acid (TCA). The samples are centrifuged at 12,000 g for 10 min. 500 μL of the supernatant is mixed with 500 μL of 0.5% (w/v) TBA and heated to 95° C. for 30 min. The absorption of TBA derivatives is measured photometrically at λ=532 nm. Blank analysis is based on emission of the mitochondria, or of FeSO₄, or alternatively, addition of the drugs after incubation.

### (d) Neuroprotective Effects

Neuroprotective effects of the iron chelators are determined both in vivo and in vitro systems.

For in vitro experiments, rat pheochromocytoma type 12 (PC12) cells and human neuroblastoma SH-SY5Y cells are used to examine the neuroprotective action of the chelators in response to iron and beta-amyloid toxicity. Cell viability is tested in 2,5-diphenyltetrazolium bromide (MTT) and lactate dehydrogenase(LDH) tests as well as measuring dopamine and tyrosine hydroxylase by HPLC and release of alpha-amyloid (soluble) by Western, since these cells are used as models of dopamine and cholinergic neurons.

The protection in vivo is tested in 1-methyl-4-phenyl-1,2,3,6-tetrahydropyridine (MPTP) animal model of Parkinson's disease (PD), a very viable and well-established model of neurodegeneration, by measuring striatal dopamine and tyrosine hydroxylase, the markers of dopamine neurons.

### (e) PC12 Cell Culture.

Rat PC12 cells, originated from rat pheochromocytoma, are grown at 37° C., in a humid 5% CO₂, 95% air environment, in a growth medium containing Dulbecco's modified Eagle's Medium (DMEM, GIBCO, BRL) supplemented with glucose (1 mg/mL), 5% fetal calf serum, 10% horse serum, and 1% of a mixture of streptomycin/penicillin. On confluence, the culture is removed and the cells are detached by vigorous washing, centrifuged at 200 g for 5 min and resuspended in DMEM with full serum content. 0.5×104 cells/well are placed in microtiter plates (96 wells) precoated with collagen.

### (f) MTT Tests for Cell Viability.

Twenty-four hours after attachment of the PC12 cells as described in (e), the medium is replaced with DMEM containing 0.1% bovine serum albumin (BSA). The test compounds are added to the cells after 1 h of incubation. After 24 h incubation, the cells are subjected to MTT test. The absorption is determined in a Perkin-Elmer Dual Wavelength Eliza-Reader at λ=570/650 nm after automatic subtraction of background readings. The results are expressed as percentage of the untreated control.

### Example 5

### The Minimal Inhibitory Concentration (MIC) of Compound 4

The minimal inhibitory concentration (MIC) of compound 4 was determined against standard strains of MDR bacteria and clinical isolates according to the guidelines of the Clinical and Laboratory Standards Institute (CLSI).

Individual MICs were determined following the microdilution method recommended by CLSI in cationic-adjusted Mueller-Hinton Broth (CAMHB), or M9 media. The MIC was defined as the lowest drug concentration that caused 100% inhibition of visible bacterial growth after 24 hours incubation. Tests were performed in triplicate. MIC of Compound 4 were determined for nosocomial ESKAPE pathogens (Enterococcus faecium, Staphylococcus aureus, Klebsiella pneumoniae, Acinetobacter baumannii, Pseudomonas aeruginosa, and Enterobacter species).

Tables l and 2 show the MIC of compound 4 determined in two different media for clinical isolates of bacterial strains of A. baumannii (AB) obtained from the Multidrug-resistant Organism Repository & Surveillance Network, USA.

Table 1 shows the MIC of compound 4 determined for clinical isolates of bacterial strains of A. baumannii in CAMHB media as described above.

TABLE 1

| Bacterial Strain | MIC of compound 4 (μg/ml) |
|---|---|
| AB967 | 500 |
| AB2828 | 500 |
| AB3340 | 500 |
| AB3560 | 500 |
| AB3638 | 500 |
| AB3785 | 500 |
| AB3806 | 1000 |
| AB3917 | 500 |
| AB3927 | 500 |
| AB4025 | 500 |
| AB4026 | 500 |
| AB4027 | 500 |
| AB4052 | 1000 |
| AB4269 | 500 |
| AB4448 | 500 |
| AB4456 | 500 |
| AB4490 | 500 |
| AB4498 | 500 |
| AB4795 | 500 |
| AB4857 | 500 |
| AB4878 | 500 |
| AB4932 | 500 |
| AB4957 | 500 |
| AB4991 | 500 |
| AB5001 | 500 |
| AB5075 | 500 |
| AB5197 | 500 |
| AB5256 | 500 |
| AB5674 | 500 |
| AB5711 | 500 |

Table 2 shows the MIC of compound 4 determined for clinical isolates of bacterial strains of A. baumannii in RPMI 1640 (low iron) media, which is closer to the iron levels in the human body.

TABLE 2

| Bacterial Strain | MIC of compound 4 (μg/ml) |
|---|---|
| AB967 | NG |
| AB2828 | NG |
| AB3340 | NG |
| AB3560 | NG |
| AB3638 | 64 |
| AB3785 | 32 |
| AB3806 | 32 |
| AB3917 | 32 |
| AB3927 | NG |
| AB4025 | 32 |
| AB4026 | 32 |
| AB4027 | 32 |
| AB4052 | 64 |
| AB4269 | 32 |
| AB4448 | 64 |
| AB4456 | 32 |
| AB4490 | 32 |
| AB4498 | 64 |
| AB4795 | 32 |
| AB4857 | 64 |
| AB4878 | 64 |
| AB4932 | 125 |
| AB4957 | 125 |
| AB4991 | 8 |
| AB5001 | 32 |
| AB5075 | 64 |
| AB5197 | 32 |
| AB5256 | 125 |
| AB5674 | 32 |
| AB5711 | 64 |

NG = no growth.

Table 2 demonstrates bacteria grown in media containing iron levels similar to the human body (low iron RPMI), the MIC for many clinical isolates of bacterial strains is greatly reduced as compared with iron containing media. For a number of strains, compound 4 produced no growth of bacteria (NG).

### Example 6

### Inhibition of Growth of A. baumannii, Strain 5711 by Compound 4 at Various Concentrations

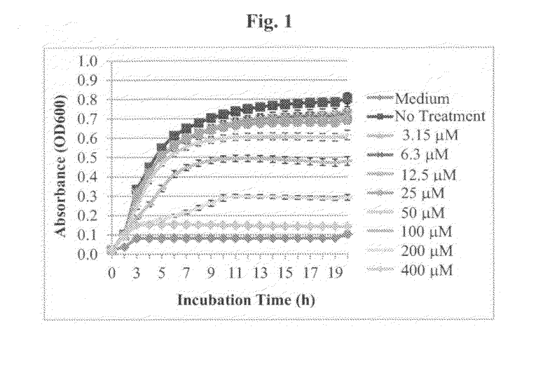

The VK-28 derivative compound 4, which is highly stable in aqueous solution, has shown an antibacterial activity against A. baumannii, see FIG. 1. For example, in growth curves of A. baumannii strain 5711 in CAMHB medium, OD₆₀₀ was dramatically reduced by the presence of compound 4. It should be noted that the inhibition occurred in a dose-responsive manner.

### Example 7

### Neuroprotection Against 6-OHDA Lesion in Rats

Significant increase in iron occurs in the substantia nigra pars compacta of Parkinsonian subjects, and in 6-hydroxydopamine (6-OHDA) treated rats and monkeys. This increase in iron has been attributed to its release from ferritin and is associated with the generation of reactive oxygen species and the onset of oxidative stress-induced neurodegeneration. D. Ben Shachar et al. (NeuroPharmacology 46 254-263. 2004) described the neuroprotection by the iron chelator Vk-28 against 6-OHDA lesioned rats.

The iron chelators 4, 5 and 6 of the present invention are examined as described in Ben Shachar et al., herewith incorporated by reference in its entirety as if described herein.

The iron chelators of the present invention (compounds 4, 5, 6) are examined for their ability to inhibit basal as well as iron-induced mitochondrial lipid peroxidation. The iron chelators are injected either intraventricularly (ICV) or intraperitoneally (IP), to 6-OHDA lesioned rats.

Male Sprague-Dawley rats (8-10 in each group) weighing 230-270 g are housed in a temperature-controlled colony room, with a 12 h light-dark cycle and free access to food and water for 4 weeks. Rats are anesthetized with a mixture of 15 mg/Kg pentobarbital and 60 mg/Kg chloral hydrate. 6-OHDA (250 μg in 5 μl of 0.9% NaCl containing 0.2% ascorbic acid), the iron chelator 4, 5 or 6 (1 μg in 5 μl) or saline is injected into the left cerebral ventricle (ICV) using stereotaxic techniques. Pargyline (50 mg/Kg i.p.) and desmethylimipramine-HCl. (25 mg/Kg i.p.) are administered to all rats 60 min before ICV injection. An additional group of rats receives the iron chelator intraperitoneally (i.p.) (1 or 5 mg/Kg/day for 10 and 7 days, respectively) or saline prior to ICV injection with 6-OHDA. All animals receive a daily injection of isotonic glucose (4 ml/day i.p.) until they regain their original body weight. The rats are sacrificed by decapitation 4 weeks after surgery, their brains are rapidly removed and the striata are dissected on ice-chilled glass plates. For monoamine determination, tissues are immediately placed in liquid nitrogen, then weighed and homogenized with 0.1N HClO4. After centrifugation the supernatant is collected and stored at −70° C. until use.

### 7.1 Measurement of Lipid Peroxidation in Brain Tissue

Cortical membrane homogenates (10% w/v in 50 mM phosphate buffer pH-7.0 containing 15 mM Na+ and 145 mM K+) from male Sprague Dawley rats are prepared. Aliquots of the cortical membrane homogenates (0.1 ml) are incubated at 37° C. for 90 min alone (basal peroxidation) or in the presence of 10⁴ M Fe³⁺. The novel iron chelator (10⁻⁷-10⁻³M) is added in the presence or absence of iron. Lipid peroxidation is estimated by measurement of malondialdehyde formation at 45° C. and expressing it per mg protein.

### 7.2. HPLC Analysis

Concentrations of norepinephrine (NA), dopamine (DA), dihydroxyphenylalanine (DOPA), dihydroxyphenylacetic acid (DOPAC), homovanillic acid (HVA), serotonin 5-HT and 5-hydroxyindolacetic acid (5-HIAA) are determined by HPLC with an electrochemical detector, equipped with a column of 5 urn spherical C18 particles. The dual electrode analytical cell is operated in a redox mode with 0.3 V oxidation potential and −0.35 V reduction potential. The mobile phase consists of 0.1 M phosphate buffer pH 2.6 containing 0.2 mM EDTA, 0.2 mM octane sulfonic acid, 2.5% methanol and 4.5% acetonitrile.

7.3 Results:

the new iron chelators of the present invention are expected to exhibit a similar or higher iron binding capacity to that of VK28 and to inhibit MDA formations with similar or higher effectivity than VK28 at similar concentrations. Striatal 5-HT, 5-HIAA and NE concentrations were not shown not be affected by both the neurotoxin and VK-28 and VK-28 had no effect on basal striatal tissue monoamine concentrations (D. B. Shachar et al., Neuropharmacology 46 (2004) 254-263) and the iron chelators of the present invention are expected to behave similarly.

### Example 8

### Neuroprotection Against Lactacystin-Induced Neurodegeneration, UPS Inhibition and Iron Elevation

Dysfunction of the ubiquitin-proteasome system (UPS) and accumulation of iron in substantia nigra (SN) are implicated in the pathogenesis of Parkinson's disease (PD). UPS dysfunction and iron misregulation may reinforce each other's contribution to the degeneration of dopamine (DA) neurons. Zhu et al. The (FASEB J. 21, 3835-3844, 2007) used the brain-permeable iron chelator, VK-28, in vivo to test its neuroprotective and neurorestorative properties against proteasome inhibitor (lactacystin)-induced nigrostriatal degeneration. Bilateral microinjections of lactacystin (1.25 μg/side) into the mouse medial forebrain bundle were performed. Administration of VK-28 (5 mg/kg, once a day) was applied intraperitoneally 7 days before or after the lactacystin microinjection until the mice were sacrificed 28 days after microinjection. It was found that VK-28 significantly improved behavioral performances and attenuated lactacystin-induced DA neuron loss, proteasomal inhibition, iron accumulation, and microglial activation in SN.

The iron chelators 4, 5 and 6 of the present invention are examined as described in Zhu et al., herewith incorporated by reference in its entirety as if described herein.

8.1 Animals—Male C57BL/6 mice aged 12 wk are randomly signed into groups: control, Lac 7d, Lac 28d, Pre-iron chelator, and Post-iron chelator. They are housed five animals per cage in a colony room maintained at constant temperature and humidity, with a 12 h light/dark cycle, and allowed at least 7 days to acclimatize before any treatment. Intraperitoneal (i.p.) administration of the iron chelator (5 mg/kg, once a day) starts 7 days before or after microinjection with lactacystin and continues until the mice are sacrificed. Administration of the same volume of saline serves as control. For stereotactic injection of lactacystin, mice are deeply anesthetized and placed in a Kopf stereotactic frame (Kopf Instruments, Tujunga, Calif., USA). An injection cannula is inserted through a hole drilled in the skull into the MFB using the following coordinates (in mm): 1.34 posterior, ±1.17 lateral, and 5.1 ventral from bregma of each mouse. Two microliters of phosphate-buffered saline (PBS, 0.1M) as control or lactacystin (1.25 μg; A.G. Scientific, San Diego, Calif., USA) in PBS is injected into the MFB of each mouse. Mice injected with lactacystin only (group Lac 7d) are sacrificed 7 days after microinjection. Other mice are sacrificed at the end of the study, 28 days after microinjection of lactacystin. The mice are sacrificed and decapitated and the brains are immediately removed, placed on ice, and transected coronally at the infundibular stem. The midbrain blocks are fixed with 4% paraformaldehyde in PBS for 2 days and cryoprotected in 30% sucrose for 2 days at 4° C., followed by histological analysis. Striatal tissues and ventral midbrains are rapidly dissected out and stored at −80° C. until analysis. The samples are divided to perform different sets of experiments.

8.2 Locomotive activities and rotarod performance are tested 1 day before and 7 and 28 days after microinjection of lactacystin. Locomotive activities are monitored by the AccuScan Digiscan system (AccuScan Instruments, Inc., Columbus, Ohio, USA). Data collected by computer included total distance traveled (cm/60 min) and moving time (s/60 min). The measurements are carried out from 9 AM to 11 AM in a dark room. Each mouse is placed in the testing chamber for 30 min for adaptation, followed by a 60 min recording by the computer-generated automatic analysis system. Motor coordination is determined with an accelerating rotarod treadmill (Columbus Instruments, Columbus, Ohio, USA). Initially, each mouse is required to perch on the stationary rod for 30 s to accustom itself to the environment. Then the animals are trained at a constant speed of 5 rpm for 90 s. After this pretraining, the mice are tested three times at 1 h intervals on 3 consecutive days for a total of nine tests. During each test, the rotarod is set at a starting speed of 5 rpm for 30 s and the speed is increased by 0.1 revolution per second. All animals are tested three times for each experiment, and the means of the test results undergo statistical analysis.

### 8.3 Immunohistochemistry

Midbrain blocks are cut into 30 μm sections and systematically picked at 150 μm intervals. Free-floating sections are incubated successively for 15 min with 0.05% H₂O₂ in 0.1 mol/L PBS to remove endogenous peroxidase activity for 1 h with 2% goat serum/0.1% Triton X-100 in 0.1 mol/L PBS to block nonspecific binding sites and for 24 h at 4° C. with primary antibodies, rabbit antityrosine hydroxylase (TH, 1:1500; Protos Biotech, New York, N.Y., USA) and mouse anti-TH (1:500; Sigma-Aldrich, St. Louis, Mo., USA), to detect DA neurons; mouse antiglial fibrillary acidic protein (GFAP, 1:500; Chemicon International Inc., Temecula, Calif., USA) to label astrocytes; rat anti-CD11b (against MAC1, 1:50; Chemicon International Inc.) to detect microglia; and mouse anti-synuclein (1:1000; BD Transduction Laboratories, San Jose, Calif., USA) and rabbit antiubiquitin (1:500; Chemicon International Inc.) to detect protein accumulations. Sections are then incubated for 2 h at room temperature with the appropriate biotinylated secondary antibody (anti-rabbit or anti-rat IgG, 1:200; Vector Laboratories Inc., Burlingame, Calif., USA). The avidin-biotin method is used to amplify the signal (ABC Kit; Vector Laboratories Inc., Burlingame, Calif., USA) and 3,3′-diaminobenzidine tetrachloride (DAB) is used to visualize bound antibodies. For double-immunofluorescent staining, Alex Fluor 546 goat anti-rabbit IgG and 488 goat antimouse IgG (1:200; Molecular Probes, Eugene, Oreg., USA) are used. The DA neurons (TH-positive cells) and microglia in the SN are counted.

### 8.4 Determination of Striatal DA and its Metabolites

The concentrations of DA, 4-dihydroxy-phenylacetic acid (DOPAC), homovanillic acid (HVA), serotonin (5-HT), and 5-hydroxyindolacetic acid (5-HIAA) are quantified in striatal tissues by HPLC. Briefly, striatal tissues are homogenized (10% w/v) by sonication in ice-cold 0.1M perchloric acid. Homogenates are centrifuged at 10,000 g for 10 min at 4° C.; the supernatants are collected and filtered through acrodisc filters (0.25 μm, Fisher Scientific) and subjected to HPLC (HTEC-500; Eicom, Kyoto, Japan) with the column (EICOMPAK SC-3ODS; Eicom, Kyoto, Japan) and detected by an electrochemical detector (AD Instruments Pty Ltd., Castle Hill, NSW, Australia). The mobile phase consists of 0.1 mM citric acid, 0.1M sodium acetate, 220 mg/L octane sulfate sodium, 5 mg/L EDTA, and 20% methanol (pH=3.5).

### 8.5 Proteasome Activity Assay

Ventral midbrains are placed on ice and homogenized in lysis buffer (50 mM HEPES, pH 7.5, 5 mM EDTA, 150 mM NaCl, 0.5 mM ATP, and 1% Triton X-100). The lysates are centrifuged at 14,000 g at 4° C. for 20 min. The resulting supernatants are placed on ice and assayed for protein concentrations by the Bradford's method (Bio-Rad, Hercules, Calif., USA). The 20S Proteasome Activity kit (Chemicon International Inc.) is used to measure chymotrypsin-like activity. Assays are carried out with 50 μg of midbrain lysates and appropriate substrate for 90 min incubation at 37° C. Activity is measured by detection of the fluorophore 7-amido-4-methylcoumarin (AMC) after cleavage from the synthetic fluorogenic peptide Leu-Leu-Val-Tyr-AMC, using a spectrofluorimeter (Cytofluor II; PerSeptive Biosystems, Framingham, Mass., USA) at excitation/emission wavelengths of 380/460 nm. Results are expressed as fluorescence units/mg protein.

### 8.6 Iron Measurement in the Ventral Midbrain

Ventral midbrains are weighed and digested in concentrated hydrochloric acid. Tissue iron concentrations (nmol/g wet tissue) are determined spectrophotometrically by using the kit from DCL (Diagnostic Chemicals Ltd., Oxford, Conn., USA) in a modified microtiter plate assay.

### 8.7 Results

The iron chelators of the invention are expected to improve behavioral performance in lactacystin-lesioned mice, to reduce lactacystin-induced loss of DA neurons in the SN, to restore lactacystin-induced depletion of DA and its metabolites, to alleviate lactacystin-induced proteasomal inhibition, to attenuate lactacystin-induced iron accumulation and to decrease microglial activation in lactacystin-injected mice, with an effect similar to VK-28 or even better.

1. A compound of the formula I: 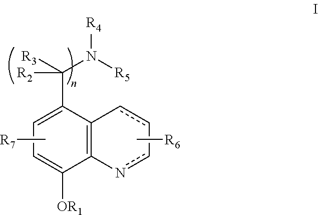
wherein
R₁ is selected from:
(i) H;
(ii) C₁-C₈ alkyl substituted by one or more radicals selected from hydroxy, C₁-C₈ alkoxy, cyano, carboxy, aminocarbonyl, C₁-C₈ alkylaminocarbonyl, di(C₁-C₈)alkylaminocarbonyl, and C₁-C₈ alkoxycarbonyl;
(iii) —COR₈, wherein R₈ is C₁-C₈ alkyl, C₂-C₈ alkenyl, C₂-C₈ alkynyl, C₃-C₈ cycloalkyl, aryl, heteroaryl, or heterocyclyl wherein said alkyl, alkenyl, alkynyl, aryl, heteroaryl, or heterocyclyl group is optionally substituted by one or more of the following groups: halogen atoms, C₁-C₈ alkyl, hydroxy, amino, C₁-C₈ alkylamino, di(C₁-C₈)alkylamino, mercapto, C₁-C₈ alkylthio, cyano, C₁-C₈ alkoxy, carboxy, C₁-C₈ (alkoxy)carbonyl, C₁-C₈ (alkyl)carbonyloxy, C₁-C₈ (alkyl)sulfonyl, C₁-C₈ (alkyl)carbonylamino, aminocarbonyl, C₁-C₈ (alkyl)aminocarbonyl, or di(C₁-C₈)alkylaminocarbonyl, or a straight or branched C₁-C₅ alkyl may be substituted by amino at the α-position to the CO group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue.
(iv) —COOR₉, wherein R₉ is C₁-C₈ alkyl optionally substituted by halogen, C₁-C₈ alkoxy, phenyl optionally substituted by nitro, hydroxy, carboxy, or C₃-C₆ cycloalkyl; C₂-C₄ alkenyl; C₂-C₄ alkynyl; C₅-C₇ cycloalkyl; or phenyl optionally substituted by halogen, amino, nitro, C₁-C₈ alkyl, C₁-C₈ (alkoxy)carbonyl, or C₁-C₈ alkoxy;
(v) —CH₂—O—CO—R₁₀, or —CH(CH₃)—O—CO—R₁₀, wherein R₁₀ is C₁-C₈ alkyl optionally substituted by halogen, C₁-C₈ alkoxy; C₂-C₄ alkenyl optionally substituted by phenyl; C₃-C₆ cycloalkyl; phenyl optionally substituted by C₁-C₈ alkoxy; or heteroaryl selected from furyl, thienyl, isoxazolyl, or pyridyl optionally substituted by halogen or C₁-C₈ alkyl;
(vi) —PO(OR₁₁)₂, —CH₂—O—PO(OR₁₁)₂ or —CH(CH₃)—O—PO(OR₁₁)₂, wherein R₁₁ is independently selected from H, C₁-C₈ alkyl, or C₁-C₈ alkyl optionally substituted by hydroxy, C₁-C₈ alkoxy, or C₁-C₈ (alkyl)carbonyloxy; and
(vii) —CONR₁₂R₁₃, wherein R₁₂ and R₁₃ are independently selected from H, C₁-C₈ alkyl, C₂-C₈ alkenyl, C₂-C₈ alkynyl, C₃-C₈ cycloalkyl, aryl, arylalkyl, heteroaryl, or heterocyclyl wherein said alkyl, alkenyl, alkynyl, aryl, heteroaryl, heteroarylalkyl, heterocyclyl or heterocyclylalkyl group is optionally substituted by one or more of the groups: halogen atoms, C₁-C₈ alkyl, hydroxy, amino, C₁-C₈ alkylamino, di(C₁-C₈)alkylamino, mercapto, C₁-C₈ alkylthio, cyano, C₁-C₈ alkoxy, carboxy, C₁-C₈ (alkoxy)carbonyl, C₁-C₈ (alkyl)carbonyloxy, C₁-C₈ (alkyl)sulfonyl, C₁-C₈ (alkyl)carbonylamino, aminocarbonyl, C₁-C₈ (alkyl)aminocarbonyl, and di(C₁-C₈)alkylaminocarbonyl, or a straight or branched C₁-C₅ alkyl may be substituted by a carboxy group at the α-position to the amino group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue. or R₁₂ and R₁₃ together with the N atom to which they are attached form a 5 to 7 membered saturated ring optionally further containing a heteroatom selected from O, S and N, optionally substituted by C₁-C₈ alkyl;
R₂ and R₃ each independently is selected from a group consisting of H, C₁-C₈ alkyl, halogen, halo(C₁-C₈)alkyl, OH, C₁-C₈ alkoxy, amino, C₁-C₈ alkylamino, di(C₁-C₈)alkylamino, C₁-C₈ (alkyl)carbonylamino, carboxy, or C₁-C₈ (alkyl)carbonyloxy;
R₄ and R₅ together with the nitrogen atom to which they are attached form a 5-8 membered heterocyclic ring that may contain one or more nitrogen, oxygen, or sulfur atoms and may be optionally substituted at any available position in the ring with one or more radicals selected from the group consisting of H, C₁-C₈ alkyl, halogen, halo(C₁-C₈)alkyl, cyano, cyano(C₁-C₈)alkyl, (C₁-C₈)alkoxy, (C₁-C₈)alkoxy(C₁-C₈)alkyl, hydroxy, hydroxy(C₁-C₈)alkyl, amino, (C₁-C₈)alkylamino, di(C₁-C₈)alkylamino, amino(C₁-C₈)alkyl, (C₁-C₈)alkylamino(C₁-C₈)alkyl, di(C₁-C₈)alkylamino(C₁-C₈)alkyl, oxo, formyl, acyl, carboxy, (C₁-C₈)alkoxycarbonyl, carboxy(C₁-C₈)alkyl, acyloxy, acyloxy(C₁-C₈)alkyl, acylamino, acylamino(C₁-C₈)alkyl, (C₁-C₈)alkylsulfonyl, and arylsulfonyl radicals;
R₆ is H, C₁-C₈ alkyl, mercapto, C₁-C₈ alkylthio, amino, C₁-C₈ alkylamino, C₁-C₈ alkylimino, di(C₁-C₈)alkylamino, hydroxy, or C₁-C₈ alkoxy; or imino, oxo or thioxo at the 2- or 4-positions;
R₇ is H, halogen, C₁-C₈ alkyl, C₃-C₈ cycloalkyl, halo(C₁-C₈)alkyl, cyano, (C₁-C₈)alkoxy, hydroxy, amino, (C₁-C₈)alkylamino, di(C₁-C₈)alkylamino, nitro, acyloxy, acylamino, (C₁-C₈)alkylthio, (C₁-C₈)alkylsulfenyl, or (C₁-C₈)alkylsulfonyl;
each of the dotted lines indicates an optional bond; and
n is an integer from 1 to 8,
and pharmaceutically acceptable salts thereof,
but excluding the compounds wherein R₁, R₂, R₃, R₆, R₇ are H; n is 1; and R₄ and R₅ together with the N atom to which they are attached form a piperazino ring substituted at the 4-position by 2-hydroxyethyl.

2. The compound according to claim 1, wherein R₁ is H.

3. The compound according to claim 1, of the formula II: 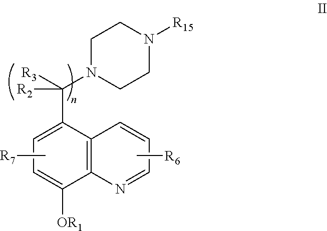
wherein
R₁, R₂, R₃ and R₇ each is as defined in claim 1;
R₆ is H, C₁-C₈ alkyl, mercapto, C₁-C₈ alkylthio, amino, C₁-C₈ alkylamino, C₁-C₈ alkylimino, di(C₁-C₈)alkylamino, hydroxy, or C₁-C₈ alkoxy;
R₁₅ is H, C₁-C₈ alkyl, halogen, halo(C₁-C₈)alkyl, cyano, cyano(C₁-C₈)alkyl, (C₁-C₈)alkoxy, (C₁-C₈)alkoxy(C₁-C₈)alkyl, hydroxy, hydroxy(C₁-C₈)alkyl, amino, (C₁-C₈)alkylamino, di(C₁-C₈)alkylamino, amino(C₁-C₈)alkyl, (C₁-C₈)alkylamino(C₁-C₈)alkyl, di(C₁-C₈)alkylamino(C₁-C₈)alkyl, oxo, formyl, acyl, carboxy, carboxy(C₁-C₈)alkyl, C₈)alkyloxycarbonyl, acyloxy, acyloxy(C₁-C₈)alkyl, acylamino, acylamino(C₁-C₈)alkyl, (C₁-C₈)alkylsulfonyl, or arylsulfonyl;
n is an integer from 1 to 8, and
pharmaceutically acceptable salts thereof, but excluding the compound wherein R₁, R₂, R₃, R₆, R₇ are H; n is 1 and R₁₅ is 2-hydroxyethyl.

4. The compound according to claim 3, wherein R₁₅ is 2-hydroxyethyl.

5. The compound according to claim 3, wherein R₁ is H, R₂, R₃, R₆ and R₇ each is as defined in claim 3, R₁₅ is 2-hydroxyethyl and n is an integer from 2 to 5, optionally 2 or 3.

6. The compound according to claim 5, wherein R₁, R₂, R₃, R₆ and R₇ each is H, R₁₅ is hydroxyethyl, and n is 2, herein identified as compound 4.

7. The compound according to claim 5, wherein R₁, R₂, R₃, and R₆ each is H, R₇ is F at the 7-position, R₁₅ is hydroxyethyl, and n is 2, herein identified as compound 5.

8. The compound according to claim 5, wherein R₁, R₂, R₃, R₆, and R₇ each is H, R₁₅ is hydroxyethyl, and n is 3, herein identified as compound 6.

9. The compound according to claim 1 or 3, wherein R₁ is selected from:
(i) C₁-C₃ alkyl substituted by hydroxy or C₁-C₃ alkoxy;
(ii) —COR₈, wherein R₈ is C₁-C₅ alkyl and said alkyl may be methyl optionally substituted by methoxy, methoxycarbonyl, methylcarbonyloxy or one or more of Cl or F atoms, or ethyl optionally substituted by ethoxy, isobutyl, or sec-pentyl; C₂-C₄ alkenyl; C₃-C₅ cycloalkyl; carbocyclic aryl, optionally substituted by methoxy; heteroaryl, optionally substituted by Cl; heterocyclyl; straight or branched C₁-C₅ alkyl substituted by amino at the α-position to the CO group, and the alkyl is optionally further substituted at a different position by hydroxy, amino, guanidino, mercapto, methylthio, carboxy, aminocarbonyl, phenyl, 4-hydroxyphenyl, 2-indolyl or 5-imidazolyl such as to form an amino acid residue derived from glycine, alanine, valine, leucine, isoleucine, serine, threonine, lysine, arginine, cysteine, methionine, aspartic, glutamic, asparagine, glutamine, phenylalanine, tyrosine, tryptophan or histidine, or the amino group and the alkyl chain form a 5-membered ring to form a proline residue;
(iii) —COOR₉, wherein R₉ is C₁-C₈ alkyl and said alkyl may be methyl optionally substituted by Cl, 4-nitrophenyl or C₆ cycloalkyl, or ethyl optionally substituted by methoxy or by one or more Cl or F atoms, propyl, butyl, isobutyl, pentyl, or octyl; C₂-C₃ alkenyl; C₃-C₄ alkynyl; C₅-C₆ cycloalkyl; or phenyl optionally substituted by nitro, fluoro, methoxy or methyl;
(iv) —CH₂—O—CO—R₁₀ or —CH(CH₃)—O—CO—R₁₀, wherein R₁₀ is C₁-C₅ alkyl and said alkyl may be methyl optionally substituted by methoxy, methoxycarbonyl, methylcarbonyloxy, or by one or more Cl or F atoms, or ethyl optionally substituted by ethoxy, isobutyl, or 1-methylbutyl; C₂-C₄ alkenyl; C₃-C₅ cycloalkyl; phenyl optionally substituted by methoxy; or heteroaryl such as 2-furyl, 2-thienyl, 5-isoxazolyl, or pyridyl optionally substituted by halogen.

10. A pharmaceutical composition comprising a compound according to any one of claims 1 to 9 or a pharmaceutically acceptable salt thereof, and a pharmaceutically acceptable carrier, but excluding the compositions when used for antibacterial therapy and treatment of wounds caused by multidrug resistant bacteria.

11. The pharmaceutical composition according to claim 10, for use in the prevention and/or treatment of neurodegenerative and cerebrovascular diseases, conditions and disorders.

12. The pharmaceutical composition according to claim 11 for use in the treatment of Parkinson's disease.

13. A compound according to any one of claims 1 to 9 or a pharmaceutically acceptable salt thereof, for use in preventing and/or treating conditions, disorders or diseases that can be prevented and/or treated by iron chelation therapy or by neuroprotective therapy.

14. The compound according to claim 13 or a pharmaceutically acceptable salt thereof, for use in the prevention and/or treatment of neurodegenerative and cerebrovascular diseases, conditions and disorders.

15. The compound according to claim 13 or a pharmaceutically acceptable salt thereof for use in treatment of Parkinson's disease.
